(19) United States
(12) Patent Application Publication
(10) Pub. No.: US 2011/0033938 A1
(43) Pub. Date: Feb. 10, 2011

(54) SYSTEM FOR INDUCIBLE GENE EXPRESSION IN CHLAMYDOMONAS

(76) Inventors: Giovanni Giuliano, Roma (IT); Paola Ferrante, Roma (IT)

Correspondence Address: NIXON & VANDERHYE, PC, 901 NORTH GLEBE ROAD, 11TH FLOOR, ARLINGTON, VA, 22203, US

(21) Appl. No.: 12/920,245

(22) PCT Filed: Feb. 25, 2009
(86) PCT No.: PCT/IT09/00064

§ 371 Date: Sep. 27, 2010

(30) Foreign Application Priority Data
Feb. 28, 2008 (IT) RM2008A000103

(51) Int. Cl. C12N 1/12; C12N 15/74

(52) U.S. Cl. 435/471; 435/257.6

(57) ABSTRACT

The unicellular green alga Chlamydomonas reinhardtii is currently used in many biotechnological applications for the production of biopharmaceuticals and of biohydrogen. These applications require the tightly controlled expression of genes. The present invention consists in the development and use of gene cassettes and culture growth media to obtain inducible, reversible expression of genes in Chlamydomonas. These cassettes contain the Chlamydomonas cytochrome c6 (Cyc6) promoter. It is already known that the Cyc6 promoter, joined to a DNA sequence, controls its transcription on the basis of the concentrations of copper (Cu2+) and nickel (Ni2+) ions in the growth medium (Quinn et al., 2003). In the present invention, the Cyc6 promoter activity was increased up to 20 times, as measured by the activity of an enzyme placed under its control, through the use of transcriptional enhancers, as for example the first intron of Rbcs2 gene and growth media with reduced amounts of transition metals and EDTA. These expedients allow to increase the induction levels of the Cyc6 promoter upon Ni2+ supplement and to induce its expression through the addition of Cu2+ specific chelators. The induction by chelators is reversible through the addition of Cu2+, allowing the expression, followed by the subsequent repression, of an exogenous gene in precise moments of Chlamydomonas cell growth cycle. The described induction followed by repression has never been described before in algal, plant or animal cells and allows the reversible expression of gene products at precise moments of the Chlamydomonas growth cycle.

### BACKGROUND ART

Chlamydomonas reinhardtii is an unicellular green alga. The recent sequencing of the C. reinhardtii genome has opened the way for post-genomic studies (Merchant et al., 2007). Chlamydomonas has a fast life-cycle, is transformable and grows in simple growth media. Thanks to the advantages listed above, Chlamydomonas is a useful system for the production of heterologous proteins of pharmaceutical value (Franklin and Mayfield, 2004) and of biohydrogen (Melis, 2007).

Promoters commonly used for heterologous expression in Chlamydomonas are constitutive promoters such as Rbcs2 (Stevens et al., 1996), Hsp70A-RbcS2 (Schroda et al., 2000) and PsaD (Fischer and Rochaix, 2001) and inducible promoters as Nit1 (induced by ammonium starvation (Ohresser et al., 1997)), Ca1 (induced by low CO₂ pressure (Villand et al., 1997)) and Cyc6 (induced by Cu depletion or by Ni or Co addition in the growth medium (Quinn et al., 2003)).

The use of inducible promoters for the production of potentially toxic gene products would allow their synthesis only after that the cell culture has reached elevated density, thus optimizing the yield. Moreover, for the production of biohydrogen, specific gene products must be expressed or silenced at precise moments during the growth cycle. It is obvious that the use of an inducible promoter has a great biotechnological potential. This promoter must give a high level of expression after the addition of an inducer and, possibly, must be switched off after the addition of an antagonist of the inducer.

An ideal inducible gene system should have the following characteristics: A) The inducer should be active at micromolar concentrations B) The induction should be reversible after the addition of micromolar concentrations of antagonist of the inducer C) The basal expression levels should be low, while the expression levels after inducer addition should be high D) The expression levels should respond quantitatively and quickly to the inducer and antagonist concentrations E) The inducer and the antagonist should not be toxic for the cell.

The Cyc6 promoter has been used to set up an inducible chloroplast gene expression system taking advantage of the high sensitivity of such promoter to Cu2+ (Surzycki et al., 2007). A limit in using this inducible system is that activation of the Cyc6 promoter is obtained through centrifugation, repeated washing and inoculation in growth media depleted of Cu2+.

It is obvious that such procedure is not applicable to large volumes, considering the technical difficulty of centrifuging algal cultures, even of few cubic meters, let alone the energy consumption for the centrifugation process.

A method to induce the transcription of the Cyc6 gene is addition of Ni2+ to the Chlamydomonas growth medium (Quinn et al., 2003). Moreover, a chelator such as EDTA, added after several hours from Ni2+ addition, has been claimed to be able to prevent the induction of this transcript. Since these studies have been performed using Northern blots, it is impossible to evaluate the effectiveness of such induction on a DNA sequence placed under the control of the Cyc6 promoter. In order to answer this question, we used the Renilla (cRLuc) luciferase (Fuhrmann et al., 2004) placed under the control of the Cyc6 promoter.

### DISCLOSURE OF THE INVENTION

In order to measure quantitatively the transcription of a gene sequence placed under the control of the Cyc6 promoter, we cloned downstream of this promoter the reporter gene cRLuc (Fuhrmann et al., 2004). This gene was also cloned downstream of the strong constitutive PsaD promoter (Fischer e Rochaix, 2001) obtaining the cassettes Cyc6:cRLuc and PsaD:cRLuc (FIG. 1A). Chlamydomonas cells were transformed with these cassettes according to Kindle (1990) and the transformed cells were inoculated in TAP growth medium (Harris, 1989) at a cell density of 10⁶ cells/ml. 30 hours later, Ni2+ was added at a final concentration of 25, 50 and 75 μM and Luc activity was measured in the cellular lysates at different times after induction using the Luciferase Assay System (Promega, cat. E2820). FIG. 1D shows that after induction with Ni2+, Luc activity is low compared to the Luc activity driven by the PsaD promoter. Moreover FIG. 1C shows that Ni2+ has toxicity effects on Chlamydomonas growth at 50 and 75 μM concentrations. Ni2+ does not have toxic effects at 25 μM, but this concentration is not sufficient to activate the Cyc6 promoter. Consequently, the inducible system described by Quinn et al (2003) does not allow to obtain high levels of induction of a reporter gene placed under the control of the Cyc6 promoter without producing toxic effects on algal cell growth.

The first intron of the RbcS2 gene is reported to increase the expression of a heterologous gene placed under its control (Lumbreras et al. 1998). This intron was cloned upstream and downstream of the Cyc6 promoter obtaining the cassettes Cyc6:Rb-int-1, Cyc6:Rb-int-2 and Cyc6:Rb-int-3 (FIG. 1B). Cells transformed with these cassettes were inoculated in TAP and TAP depleted of Cu2+ (Quinn and Merchant, 1995) at cell density of 10⁶ cells/ml. Luc activity was measured 48 hours after the inoculum (Table 1). The constructs Cyc6:Rb-int-1 and Cyc6:Rb-int-2, containing the RbcS2 intron, show an increased Luc activity in the growth medium depleted of Cu2+, respectively of 13 (Cyc6:Rb-int1) and 4 (Cyc6:Rb-int2) times more than the Cyc6:cRLuc construct. The Cyc6:Rb-int3 construct shows a Luc activity comparable to the Cyc6:cRLuc construct. Therefore the addition of an enhancer element upstream and downstream the Cyc6 promoter is a new method to increase its expression and it is claimed in the present invention.

In a second version of this invention the content of transition metals and EDTA in TAP growth medium was systematically modified. The reason for that is that the stock solution containing the transition metals (Hutner et al., 1950) used to prepare TAP growth medium was developed fifty years ago for bacterial growth and until now has not been optimized for Chlamydomonas growth and metabolism (Merchant et al., 2006). The minimal concentrations of transition metals able to support Chlamydomonas growth are probably lower than those present in the TAP growth medium (Merchant et al., 2006). Since transition metals and EDTA could antagonize Ni2+ action, their reduction in the growth medium could increase Cyc6 activation by this ion. As an example, the concentrations of transition metals and EDTA of the TAP growth medium and of two different growth media developed in the present invention are shown in Table 2: the growth medium TAP/ENEA1 is identical to TAP, except for Cu2+ concentration, that is 0.3 μM instead of 6 μM. The growth medium TAP/ENEA2 has reduced amounts of all transition metals and of the chelator EDTA.

Ni2+ was added to cells transformed with the construct Cyc6:cRLuc (indicated as “transformed cells” from now on) and grown in TAP, TAP/ENEA1 and TAP/ENEA2 growth media. FIG. 2 shows the Luc activity and growth curves of cultures treated with 25 (panels A and B), 50 (panels C and D) and 75 μM Ni2+ (panels E and F). At 25 μM Ni2+, the Cyc6 promoter is active only in TAP/ENEA2 growth medium, reaching the same level of activity as the strong PsaD promoter (as a reference for PsaD activity, aliquots of transformed cells collected at 40 hours for the inoculum showing an activity of 0.4 CPS/cell were used, see FIG. 1D). At 50 μM Ni2+, Cyc6 promoter activity is maximum in TAP/ENEA2 growth medium (2.5 fold more than PsaD) and lower in TAP/ENEA1 (½ PsaD) and in TAP ( 1/10 PsaD). At 50 μM Ni2+, Cyc6 promoter activity is increased 25 times in TAP/ENEA2 growth medium with respect to TAP medium. Luc activity induction at 75 μM Ni2+ is very similar in media TAP/ENEA1 and TAP/ENEA2 media (about two-fold PsaD) and lower in TAP growth medium (similar to PsaD activity). As it is evident from the growth curves in FIGS. 2B, 2D and 2F both TAP/ENEA1 and TAP/ENEA2 media support vigorous growth of Chlamydomonas cells. Ni2+ addition at 25 μM in TAP/ENEA2 growth medium has only minor toxic effects in all three growth media tested, while it causes an evident slowing down of Chlamydomonas growth rate at 50 and 75 μM in all three growth media tested. An ideal inducible gene system should be promptly reversible after the addition of an antagonist of the inducer, effective at micromolar concentrations. Using Northern blotting, Quinn et al. (2003) showed that EDTA, added 5 hs after Ni addition, is able to prevent induction of Cyc6 transcript levels by Ni. Since, at this time, the Cyc6 transcript is still undetectable, this can not formally be considered as a reversion, but rather as a lack of induction.

Indeed, the experiment shown in FIG. 3 shows that Luc activity can only be partially reverted by EDTA addition. In this experiment, the Cyc6 promoter was induced by 25 μM and 50 μM Ni2+ and 16 hours after Ni2+ addition, EDTA was added at the final concentration of 25, 50 and 150 μM. At all the EDTA concentrations tested, Cyc6 promoter activity is only partially reverted by EDTA addition both in TAP/ENEA2 (FIGS. 3A and 3B) and in TAP (FIG. 3C) growth media. Thus, the reversible method based on Ni2+/EDTA addition proposed by Quinn et al. (2003) can not be used to express reversibly a protein placed under the control of the Cyc6 promoter.

This result can be rationally explained considering that Ni2+ binds irreversibly to the transcriptional regulator CRR1 (Kropat et al., 2005), and that EDTA can not sequester all the Ni2+ present in the growth media due to the high concentrations used to obtain Cyc6 activation. Since the Cyc6 promoter is switched off by Cu2+, the use of chelators specific for that ion can be an alternative method for the reversible induction of this promoter. Some chelators specific for Cu2+ are listed in Table 3. As an example, FIGS. 4A and 4B show, respectively, the Luc activity and the growth curves of cells transformed with the construct Cyc6:cRLuc grown in TAP/ENEA2 growth medium and induced with the chelators listed in Table 3. The growth curves (FIG. 4B) show that only 1.10 phenanthroline is toxic at 10 μM, while all the other chelators tested do not have negative impact on Chlamydomonas growth. TETA 2 μM and 10 μM, BCS and IM 10 μM are able to activate the Cyc6 promoter (FIG. 4A). The more effective chelator in sequestering Cu2+ is TETA, and therefore further experiments have been carried out only with that compound. Comparative analyses of Cyc6 promoter induction by TETA supplement carried out on cells grown in TAP/ENEA2 and TAP show that the Cyc6 promoter is activated by 2 and 10 μM TETA only in TAP/ENEA2 growth medium (data not shown). This result can be explained considering that TETA is highly selective for Cu2+ and that TAP growth medium has 6 μM Cu2+ vs 0.3 μM of TAP/ENEA2 growth medium. Since the Cyc6 promoter is sensitive to nanomolar levels of Cu2+, TETA can not sequester completely all the Cu2+ present in TAP growth medium thus allowing Cyc6 activation. These results confirm the usefulness of growth media with reduced transition metal concentrations for optimizing Cyc6 promoter induction, and moreover indicate an alternative method to carry out the above mentioned induction. In the present invention, we propone an alternative method to the Ni2+/EDTA system: activation by Cu chelators, followed by reversion by Cu2+ addition. As shown in FIG. 4C, contrary to the Ni2+/EDTA system, induction by TETA can be reverted by the addition of micromolar concentration of Cu2+. Luc activity increases steadily until 48 hours after chelator addition, while addition of 2 μM Cu2+ 16 hours after chelator addition represses Luc activity with kinetics very similar to the calculated half life of Luc protein (2 hours, Fuhrmann et al. 2004). In this way a peak of gene expression is obtained, where gene activation is mediated by TETA while its repression by Cu2+. This peak can be induced and subsequently repressed for several cycles. As an example, FIGS. 4D and 4E show activation and repression mediated respectively by TETA and by Cu2+ for two consecutive growth cycles. In these experiments, the Cyc6 promoter was induced by 2 μM TETA and repressed by 1 μM Cu2+ (FIG. 4D) and by 5 μM TETA and by 2 μM Cu2+ (FIG. 4E). At 88 hours from TETA addition, the culture in stationary phase was diluted 1:20 and after 30 hours (0 hours of the second cycle) TETA was added at concentrations of 2 (FIG. 4D) e 5 μM (FIG. 4E). At 16 hours Cu2+ was added at the concentration of 1 (FIG. 4D) and 2 μM (FIG. 4E). It is evident that in the second cycle, a second induction is obtained. The priority search we did indicates that it is the first time that such a reversible gene regulation system is described in algal, plant and mammalian cells. In the system described here, activation and repression of the promoter are obtained respectively by addition of an inducer and its antagonist. In none of the chemically regulated gene expression systems described to date, such an activation followed by repression has been described.

### Description of the Gene Cassettes

The Cyc6 promoter and its 5′UTR (from −852 to +79 with respect to the transcription starting site) (Quinn and Merchant, 1995) was cloned upstream of a polylinker. The PsaD terminator (Fischer e Rochaix, 2001) was cloned downstream of this polylinker. The polylinker sequence (shown in bold) flanked at the 5′ by Cyc6 promoter sequence and at 3′ by the PsaD terminator sequence is as follows:

TCCATCGACTCTAGAGGCCTGCGGCCGCCTGCAGACTAGTAGATCT
Cyc6 XbaI StuI NotI PstI SpeI BglII
GAATTCGGCCGGCCCTAGATGGCAGCAGC
EcoRI FseI PsaD

The restriction sites present in the polylinker sequence allow cloning of a DNA sequence between the Cyc6 promoter and the PsaD terminator. In the example shown here, the cRLuc gene was cloned in the XbaI and BgIII sites.

### Tables:

TABLE 1
Luc activity of cells transformed with Cyc6:cRLuc, Cyc6:Rb-
int-1, Cyc6:Rb-int-2, Cyc6:Rb-int-3 and PsaD:cRLuc constructs
in TAP supplemented and depleted of Cu2+

| TAP | Cyc6:cRLuc | Cyc6:Rb-int-1 | Cyc6:Rb-int-2 | Cyc6:Rb-int-3 | PsaD:cRLuc |
|---|---|---|---|---|---|
| −Cu | 1* | 13 | 4 | 1 | 14 |
| +Cu | 1* | 3 | 2 | 2 | 728 |

Luc activity of Cyc6:cRLuc construct was referred as 1

TABLE 2
Element concentration in TAP, TAP/ENEA1 e TAP/ENEA2 (μM)

| | TAP | TAP/ENEA1 | TAP/ENEA2 |
|---|---|---|---|
| Zn | 77 | 77 | 3 |
| Mn | 26 | 26 | 3 |
| Fe | 18 | 18 | 5 |
| Co | 7 | 7 | 0.1 |
| Cu | 6 | 0.3 | 0.3 |
| Mo | 1 | 1 | 0.1 |
| EDTA | 134 | 134 | 15 |

TABLE 3
Characteristics of some of the Cu2+ chelators tested

| Abbreviation | Full name | Note | Reference |
|---|---|---|---|
| PHE | 1,10-phenanthroline | Cu-chelator | (Bardsley et |
| | | | al., 1974) |
| CDTA | trans-1,2-diaminocyclohexane- | Cu-chelator | (Sanchez et |
| | N,N,N9,N9-tetraacetic acid | Remedy in metal | poisoning |
| | | poisoning | al., 1994) |
| TETA | triethylentetramine | Cu-chelator. | (Cohen et |
| | | Remedy in Wilson's | al., 1983) |
| | | disease | |
| DDC | sodium diethyldithiocarbamate | Cu-chelator | (Bardsley et |
| | | | al., 1974) |
| DPA | D-penicillamine | Cu-chelator. | (Cohen et |
| | | Remedy in Wilson's | al., 1983) |
| | | disease | |
| BCS | bathocuproinedisulfonic acid | Cu-chelator | (Li and Trush, |
| | | | 1993) |
| IM | 1,3,5-cis,cis,-triaminocyclohexane- | Cu-chelator | Ma et |
| | N,N′,N″-tris-(2-methyl-(N- | | al., 2002 |
| | methylimidazole)) | | |
| DOTA | 1,4,7,10-Tetraazacyclododecane- | Cu-chelator | Boswell et |
| | 1,4,7,10-tetraacetic acid | radioactive | al., 2004 |

### Legends to Figures

FIG. 1: Comparative Expression of PsaD and Cyc6 Promoters in Tap Medium

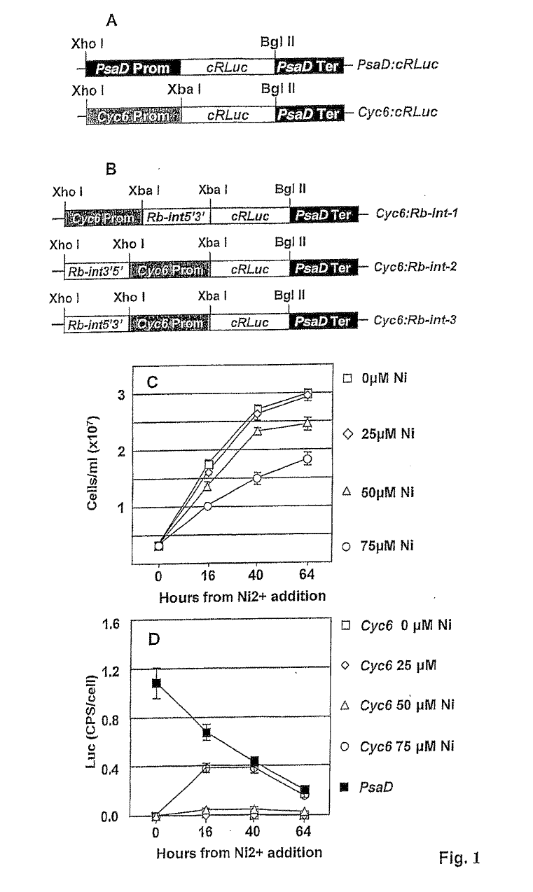

A: Schematic maps of the PsaD:cRLuc and Cyc6:cRLuc constructs
B: Schematic maps of the Cyc6:Rb-int-1, Cyc6:Rb-int-2, Cyc6:Rb:int-3 constructs
C: Growth curves in TAP medium after addition of different Ni concentrations
D: Luc activity driven by PsaD and Cyc6 in TAP medium after addition of different Ni concentrations

FIG. 2: Effect of modified TAP media on Chlamydomonas growth and on Cyc6 Promoter Expression: TAP, TAP/ENEA1 and TAP/ENEA2.

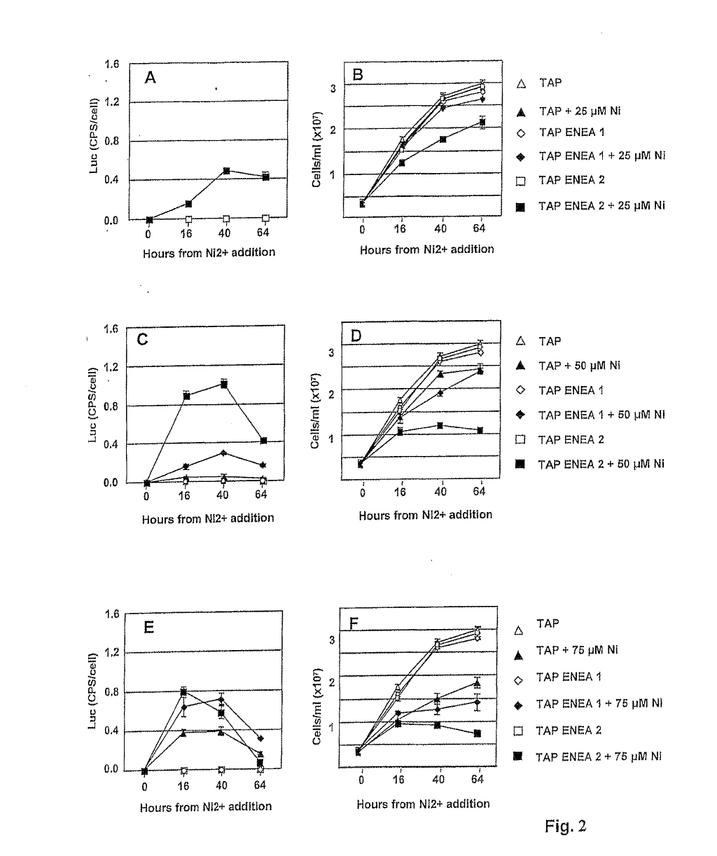

A, C, E: Luc activity of Chlamydomonas cells transformed with the Cyc6:cRLuc construct in TAP, TAP/ENEA1 and TAP/ENEA2 growth media in the presence of 25 μM (A), 50 μM (C), 75 μM (E) Ni2+.
B, D, F: Growth curves of the transformed cells grown in TAP, TAP/ENEA1 and TAP/ENEA2 growth media in the presence of different Ni2+ concentrations, 25 μM (B), 50 μM (D), 75 μM (F) Ni2+. For the composition of the different media, see Table 2.

FIG. 3: Reversibility of Ni2+/EDTA Induction in the Growth Media Tap/ENEA2 and TAP.

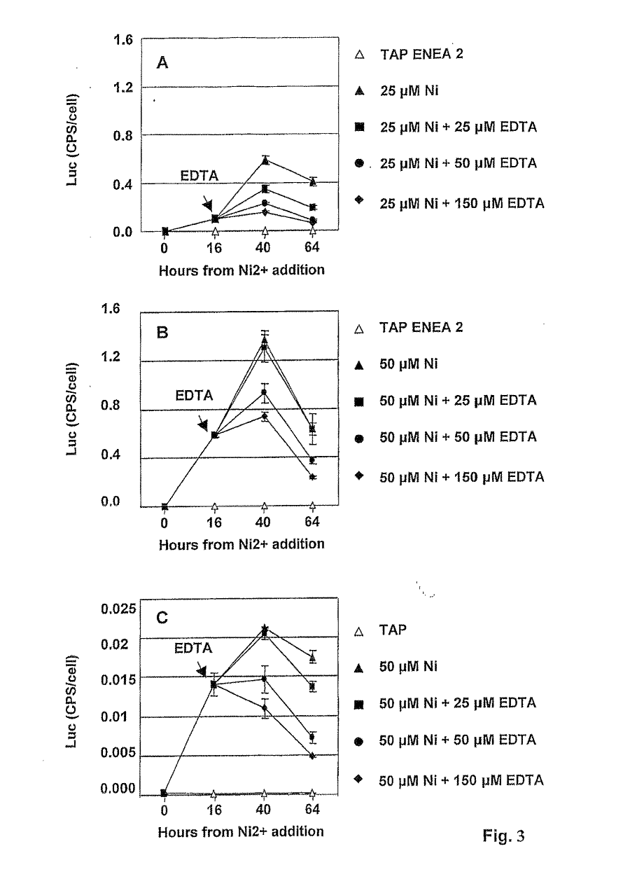

A: Luc activity of the cells transformed with the Cyc6:cRLuc construct grown in TAP/ENEA2 and induced with 25 μM Ni2+. 16 hs after Ni2+ induction, EDTA was added at the final concentrations of 25, 50 and 150 μM. The arrow indicates when EDTA was added.
B: As in FIG. A, in cultures grown in TAP/ENEA2 and induced with 50 μM Ni2+.
C: As in FIG. A, in cultures grown in TAP and induced with 50 μM Ni2+.

FIG. 4: Reversible Induction of the Cyc6 Promoter by Different Cu Chelators in TAP/ENEA2 Growth Medium.

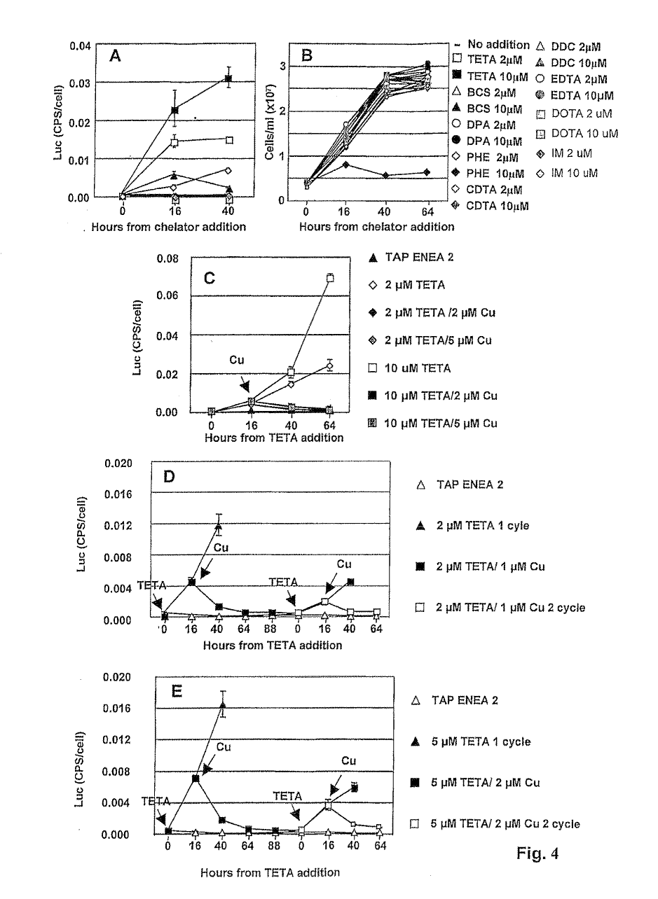

A: Luc activity of cells transformed with Cyc6:cRLuc construct induced by the different chelators listed in Table 3 in TAP/ENEA2 growth medium.
B: Growth curves in TAP/ENEA2 growth medium in the presence of different Cu-chelators. The complete name and references for each chelator are reported in Table 3. Error bars have been omitted for clarity and are below 7% respect to the average value.
C: Luc activity of cells transformed with Cyc6:cRLuc construct and induced with 2 and 10 μM TETA. 16 hours after TETA addition, Cu was added at the final concentrations of 2 and 5 μM. The arrow indicates when Cu2+ was added.
D and E: Luc activity of cell transformed with Cyc6:cRLuc construct induced with 2 μM (D) and 5 μM TETA (E), monitored for two subsequent cycles. For each cycle at 16 hs after TETA addition, Cu2+ was added at the final concentrations of 1 μM (D) and 2 μM (E). The arrows indicate when TETA and Cu2+ were added.

### REFERENCES

Bardsley, W. G., Childs, R. E., and Crabbe, M. J. (1974). Inhibition of enzymes by metal ion-chelating reagents. The action of copper-chelating reagents on diamine oxidase. Biochem J 137, 61-66.
Boswell, C. A., Sun, X., Niu, W., Weisman, G. R., Wong, E. H., Rheingold, A. L., Anderson, C. J. (2004). Comparative in vivo stability of copper-64-labeled cross-bridged and conventional tetraazamacrocyclic complexes. J. Med. Chem. 47, 1465-1474.
Cohen, N. L., Keen, C. L., Lonnerdal, B., and Hurley, L. S. (1983). The effect of copper chelating drugs on liver iron mobilization in the adult rat. Biochem Biophys Res Commun 113, 127-134.
Fischer, N., and Rochaix, J. D. (2001). The flanking regions of PsaD drive efficient gene expression in the nucleus of the green alga Chlamydomonas reinhardtii. Mol Genet Genomics 265, 888-894.
Franklin, S. E., and Mayfield, S. P. (2004). Prospects for molecular farming in the green alga Chlamydomonas. Curr Opin Plant Biol 7, 159-165.
Fuhrmann, M., Hausherr, A., Ferbitz, L., Schodl, T., Heitzer, M., and Hegemann, P. (2004). Monitoring dynamic expression of nuclear genes in Chlamydomonas reinhardtii by using a synthetic luciferase reporter gene. Plant Mol Biol 55, 869-881.
Harris, E. H. (1989). The Chlamydomonas sourcebook: A comprehensive guide to biology and laboratory use. (San Diego: Academic Press).
Hutner, S. H., Provasoli, L., Schatz, C. P., and Haskins, C. P. (1950). Some approaches to the study of the role of metals in the metabolism of microorganisms. Proc. Am. Philos. Soc. 94, 152-170.
Kindle, K. L. (1990). High-frequency nuclear transformation of Chlamydomonas reinhardtii. Proc Natl Acad Sci USA 87, 1228-1232.
Kropat, J., Tottey, S., Birkenbihl, R. P., Depege, N., Huijser, P., and Merchant, S. (2005). A regulator of nutritional copper signaling in Chlamydomonas is an SBP domain protein that recognizes the GTAC core of copper response element. Proc Natl Acad Sci USA 102, 18730-18735.
Li, Y., and Trush, M. A. (1993). Oxidation of hydroquinone by copper: chemical mechanism and biological effects. Arch Biochem Biophys 300, 346-355.
Lumbreras, V., Stevens, D. R., and Purton, S. (1998). Efficient foreign gene expression in Chlamydomonas reinhardtii mediated by an endogenous intron. Plant J 14, 441-447.
Ma, D., Lu, F., Overstreet, T., Milenic, D. E., Brechbiel, M. W. (2002). Novel chelating agents for potential clinical applications of copper. Nuclear Medicine and Biology, 29, 91-105.
Melis, A. (2007). Photosynthetic H2 metabolism in Chlamydomonas reinhardtii (unicellular green algae). Planta 226, 1075-1086.
Merchant, S. S., Allen, M. D., Kropat, J., Moseley, J. L., Long, J. C., Tottey, S., and Terauchi, A. M. (2006). Between a rock and a hard place: trace element nutrition in Chlamydomonas. Biochim Biophys Acta 1763, 578-594.
Merchant, S. S., et al. (2007). The Chlamydomonas genome reveals the evolution of key animal and plant functions. Science 318, 245-250.
Ohresser, M., Matagne, R. F., and Loppes, R. (1997). Expression of the arylsulphatase reporter gene under the control of the nit1 promoter in Chlamydomonas reinhardtii. Curr Genet. 31, 264-271.
Quinn, J. M., and Merchant, S. (1995). Two copper-responsive elements associated with the Chlamydomonas Cyc6 gene function as targets for transcriptional activators. Plant Cell 7, 623-628.

Quinn, J. M., Kropat, J., and Merchant, S. (2003). Copper response element and Crr1-dependent Ni(2+)-responsive promoter for induced, reversible gene expression in Chlamydomonas reinhardtii. Eukaryot Cell 2, 995-1002.
Sanchez, D. J., Colomina, M. T., Domingo, J. L., Llobet, J. M., and Corbella, J. (1994). Developmental toxicity of cyclohexanediaminetetraacetic acid (CDTA) in mice. Res Commun Chem Pathol Pharmacol 83, 329-340.
Schroda, M., Blocker, D., and Beck, C. F. (2000). The HSP70A promoter as a tool for the improved expression of transgenes in Chlamydomonas. Plant J 21, 121-131.
Stevens, D. R., Rochaix, J. D., and Purton, S. (1996). The bacterial phleomycin resistance gene ble as a dominant selectable marker in Chlamydomonas. Mol Gen Genet. 251, 23-30.
Surzycki, R., Cournac, L., Peltier, G., and Rochaix, J. D. (2007). Potential for hydrogen production with inducible chloroplast gene expression in Chlamydomonas. Proc Natl Acad Sci USA.
Villand, P., Eriksson, M., and Samuelsson, G. (1997). Carbon dioxide and light regulation of promoters controlling the expression of mitochondrial carbonic anhydrase in Chlamydomonas reinhardtii. Biochem J 327 (Pt 1), 51-57.

1. Modified growth media, sustaining the growth of Chlamydomonas reinhardtil cells and method for inducing the expression in said cells of a protein encoded by a gene cassette placed under the control of the Cyc6 promoter, through the addition of a chemical to the medium.

2. Method of claim 1, wherein the chemical is Ni2+.

3. Method of claim 1, where the chemical is a non-toxic, copper-specific chelator.

4. Method to repress the induction of claim 3 through the addition of Cu2+.

5. Method combining the induction by addition of a non-toxic, copper-specific chelator, followed by the repression by addition of Cu2+ to induce the temporally transient expression of a protein as claimed in claim 1.

6. Method to induce a series of transient expressions as claimed in claim 5, through a series of inductions by addition of a non-toxic, copper-specific chelator each followed by repression by addition of Cu2+.


SEQUENCE LISTING (1 sequence)

SEQ ID NO: 1 - DNA, 75 bp, Artificial Sequence
  synthetic polylinker
tccatcgactctagaggcctgcggccgcctgcagactagtagatctgaattcggccggccctagatggcagcagc